(19) United States
(12) Patent Application Publication
(10) Pub. No.: US 2007/0265354 A1
(43) Pub. Date: Nov. 15, 2007

(54) Silicon Structure

(76) Inventor: Leigh Trevor Canham, Worcestershire (GB)

Correspondence Address: NIXON & VANDERHYE, PC, 901 NORTH GLEBE ROAD, 11TH FLOOR, ARLINGTON, VA, 22203, US

(21) Appl. No.: 11/665,979

(22) PCT Filed: Oct. 19, 2004
(86) PCT No.: PCT/GB05/04027

§ 371 Date: Jun. 19, 2007

(30) Foreign Application Priority Data
Oct. 21, 2004 (GB) 0423383.9

(51) Int. Cl. A61F 2/02

(52) U.S. Cl. 514/770; 424/422

(57) ABSTRACT

The invention concerns a silicon structure comprising a metallic component and a silicon component, the metallic component and the silicon component being arranged such that at least part of the silicon component is in electrical contact with at least part of the metallic component, characterised in that the silicon component comprises nanocrystalline silicon and the metallic component comprises nanocrystalline metal. The silicon structure may be used for medical applications.

The present invention relates to a silicon structure comprising a silicon component and a metallic component. More specifically the invention relates to a silicon structure, comprising a silicon component and a metallic component, for use as a biomaterial.

A biomaterial is a non-living material suitable for use in or on the surface of a living human or animal body. When introduced into or onto a subject, a biomaterial interacts with its biological environment; for example biomaterials may be bioinert, bioactive, or resorbable, depending on their behaviour in living tissue.

Porous and polycrystalline silicon each exhibit biomaterial properties. Typically mesoporous silicon is resorbable, microporous silicon is bioactive, and macroporous silicon is bioinert. Bulk crystalline silicon is also relatively bioinert, and is present in many medical implants, in the form of electronic components. Because it is not erodable, if implantation of a bulk crystalline silicon implant has involved surgery, further surgery may be required to remove the implant once treatment has run its course.

Silicon has a number of other properties that contribute to its value for use in medical devices. For example fibres and membranes may be fabricated from porous silicon, and these have been shown to have varying degrees of mechanical flexibility. Porous silicon is generally more flexible than bulk crystalline silicon. However silicon, including porous silicon, is also relatively brittle, lacking ductility required for many applications. These mechanical properties are particularly relevant with regard to the fabrication of medical devices, since human and animal bodies contain many fibres, membranes, and tissues that are flexible and ductile.

Although silicon is generally regarded as a brittle material, a brittle-ductile phase transition has been reported for silicon at 500 C.-1000 C. associated with increasing plastic flow (Properties of Crystalline Silicon EMIS Data Review No 20 Inspec 1999). Sufficient pressure has been shown theoretically to induce phase changes to a metallic “beta-tin” form (Yin & Cohen Phys Rev Lett 45,12 p1004 (1980)) and experimentally to induce ductile behaviour, during nanoindentation, at room temperature (Gogotsi et al Semicond. Sci. Tech. 14, 93644 (1999), Domnich et al Appl. Phys. Lett 76, 2214 (2000)). Such phase changes have been observed in hydrostatically compressed porous silicon, using a diamond anvil cell and methanol/ethanol as the compressing medium (Deb et al Nature 414, 528 (2001)).

Resorbable, non-silicon containing, alloys have been developed for stent applications. For example, magnesium alloys are presently under clinical evaluation. However, magnesium has a low ductility and its use may result in the formation of fragments that can be released into the bloodstream.

The following patent documents provide background information that is relevant to the present invention: US 20040098108 describes the use of magnesium alloys for medical applications; U.S. Pat. No. 6,287,332 describes the use of different combinations of metals for medical applications; US 20030221307 describes a process for realizing thin walled tubular magnesium implants; US 20020183829 describes stent materials; US 20040002752 describes a sacrificial anode stent system whereby galvanic effects are utilised to minimize corrosion of part of the stent at the expense of another part; U.S. Pat. No. 5,855,599 describes the use of a micromachined monocrystalline disk with spring loaded helical wings as a stent; US 20030187496 describes the use of diamond like nanocompsite layers as expandable coatings for stents, WO 9706101 describes bioactive and resorbable forms of silicon, WO 03055534 describes silicon fibres and fabrics, and US 20040161369 A1 describes metal impregnated porous substrates.

The following patent documents are also relevant to the present invention: U.S. Pat. No. 5,837,030; US 2004/0129112; U.S. Pat. No. 5,964,965; U.S. Pat. No. 5,147,449; CA 2 441 578; and DE 19708402. US 2004/0129112 concerns nanocrystalline materials comprising a metal. U.S. Pat. No. 5,837,030 describes a method for preparing a nanocrystalline powder. U.S. Pat. No. 5,964,965 describes a material for gas phase hydrogenation. U.S. Pat. No. 5,147,449 describes a process for producing a powder comprising a metal matrix. DE19708402 describes a nanocrystalline material for use in engines. CA 2 441 578 describes a material comprising metal grains and ceramic grains.

Further items of prior art that provide relevant background information include: Stark et al in Proc 39^{th} Intern. Reliability Physics Symp. Florida USA IEEE p112-119 (2001) which describes silicon-glass micropackages; Journal of Alloys and Compounds 264 (1998) 285-292 which describes microstructural investigations of materials; Thin Solid Films 320 (1998) 184-191 which describes microstructure of nanocomposites; Materials Science and Engineering B100 (2003) 27-34 which describes the preparation of nanoaggregates by a thermal activation reaction; Catalysis Today 26 (1995) 247-254 which describes the development of stable supports for high temperature combustion catalysts; and Composite Science and Technology 64 (2004) 1895-1898 which describes thermal expansion and thermal mismatch stresses in a composite.

It is an objective of the present invention to at least partly solve at least some of the above mentioned problems. It is a further objective of the invention to provide a silicon structure that has improved mechanical properties. It is a yet further aspect of the present invention to provide a silicon structure that has improved resorption properties.

According to a first aspect the invention provides a silicon structure comprising a silicon component and a metallic component, the silicon component and the metallic component being arranged such that there is electrical contact between at least part of the silicon component and at least part of the metallic component.

Contact of the silicon structure with a physiological electrolyte may cause a potential difference to be established between the silicon component and the metallic component. The potential difference, resulting from the electrochemical interaction of the electrolyte with the metal and silicon, may affect erosion of the silicon component and metallic component in the electrolyte.

The silicon component may comprise one or more of: bulk crystalline silicon, porous silicon, polycrystalline silicon, amorphous silicon, nanoparticulate silicon, microparticulate silicon, nanocolumnar silicon, microcolumnar silicon, silicon having a tetrahedral bonding structure, and silicon having a beta tin crystal structure.

For the purposes of this specification nanoparticulate material is a material that comprises a multiplicity of material nanoparticles, each material nanoparticle comprising the material and having a largest dimention less than 10 nm; a nanocolumnar material is a material that comprises a multiplicity of material nano-columns, each material nano-column comprising the material and having a mean column diameter less than 10 nm; a nanostructured material is a material that comprises nanoparticles and/or nanocolumns; and a nanocomposite is a composite that comprises two or more nanostructured materials.

For the purposes of this specification microparticulate material is a material that comprises a multiplicity of material microparticles, each material microparticle comprising the material and having a largest dimention less than 10 micrometres; a microcolumnar material is a material that comprises a multiplicity of material micro-columns, each material micro-column comprising the material and having a mean column diameter less than 10 micrometres; a microstructured material is a material that comprises microparticles and/or microcolumns; and a microcomposite is a composite that comprises two or more microstructured materials.

The silicon structure may comprise a unitary body. The silicon structure may form part of a unitary body.

For the purposes of this specification a unitary body that has a structure and composition such that any part of the unitary body is integral with the other part or parts of the unitary body.

At least part of the metallic component may be integral with at least part of the silicon component.

The silicon component may comprise a multiplicity of bonded silicon particles, each bonded silicon particle being bonded to one or more of the other bonded silicon particles.

The silicon component may comprise a multiplicity of covalently bonded silicon particles, each covalently bonded silicon particle being covalently bonded to one or more of the other covalently bonded silicon particles.

The metallic component may comprise a multiplicity of bonded metal particles, each bonded metal particle being bonded to one or more of the other bonded metal particles.

The silicon structure may comprise a multiplicity of bonded particles, each bonded particle being bonded to at least one of the other bonded particles.

The silicon component may comprise semiconductor silicon. The silicon component may comprise semiconductor porous silicon. The silicon component may comprise semiconductor nanoparticulate and/or nanocolumnar silicon. The silicon component may comprise semiconductor bulk crystalline silicon.

The silicon component may comprise elemental silicon. The silicon component may comprise elemental porous silicon. The silicon component may comprise elemental nanoparticulate and/or nanocolumnar silicon. The silicon component may comprise elemental bulk crystalline silicon.

The silicon component may comprise one or more of macroporous silicon, mesoporous silicon, and microporous silicon.

Microporous silicon contains pores having a diameter less than 20 A; mesoporous silicon contains pores having a diameter in the range 20 A to 500 A; and macroporous silicon contains pores having a diameter greater than 500 A.

The silicon component may comprise porous silicon at least part of the metallic component being located in at least some of the pores of the porous silicon.

The silicon component may comprise porous silicon having a beta tin structure, at least part of the metallic component being located in at least some of the pores of the porous silicon.

The silicon component may comprise porous silicon having a tetrahedral bonding structure. The silicon component may comprise bulk crystalline silicon having a tetrahedral bonding structure.

The metallic component may comprise a metal that is more noble than silicon. The metallic component may comprise one or more of: gold, platinum, silver, palladium, selenium, copper, bismuth, tungsten, molybdenum, nickel, and iron.

The metallic component may comprise a metal that is less noble than silicon. The metallic component may comprise one or more of: aluminium, titanium , manganese, calcium and magnesium.

The metallic component may comprise a metal that is resorbable in physiological liquids. The metallic component may comprise one or more of iron, magnesium, and zinc.

The metallic component may be selected from one or more of: cobalt, chromium and vanadium.

The metallic component may comprise a multiplicity of metal wires. The metallic component may comprise a multiplicity of metal fibres. The metallic component may comprise a metal mesh. The metallic component may comprise a multiplicity of metal particles. The metallic component may be substantially uniformly distributed though the silicon component.

The metal component may comprise between 10² and 10¹⁸ metal particles. The metal component may comprise between 10⁸ and 10¹⁶ metal particles. The metal component may comprise between 10⁹ and 10¹⁴ metal particles.

The metallic component may comprise a nanoparticulate and/or nanocolumnar metal.

The metallic component may comprise a porous metal, at least part of the silicon component being located in at least some of the pores of the porous metal.

The metallic component may comprise a porous metal, and the silicon component may comprise silicon having a beta tin structure, at least part of the silicon component being located in at least some of the pores of the porous metal.

The metallic component may form greater than 30 atomic percent of the silicon structure. The metallic component may form greater than 50 atomic percent of the silicon structure. The metallic component may form greater than 70 atomic percent of the silicon structure.

The silicon component may form less than 70 atomic percent of the silicon structure. The silicon component may form less than 50 atomic percent of the silicon structure. The silicon component may form less than 30 atomic percent of the silicon structure.

The silicon component may form greater than 70 atomic percent of the silicon structure. The silicon component may form greater than 50 atomic percent of the silicon structure. The silicon component may form greater than 30 atomic percent of the silicon structure.

The metallic component may comprise a nanostructured metal and the silicon component may comprise nanostructured silicon, at least part of the nanostructured silicon being in electrical contact with at least part of the nanostructured metal.

Certain forms of porous and polycrystalline silicon, which have favourable biological properties, are nanostructured. The presence of the nanostructured silicon may confer bioactivity and/or resorbability to the silicon structure. The presence of the nanostructured metal may confer ductility to the silicon structure, and allow it to be employed in the fabrication of medical devices that require ductility for their function.

The silicon structure may have a composition such that, and the metallic component and silicon component may each be arranged such that, the silicon structure exhibits a total permanent strain up to fracture of greater than 5%.

The metallic component and silicon component may each be arranged and present in amounts such that the silicon structure exhibits a total permanent strain up to fracture of greater than 10%.

The metallic component and silicon component may each be arranged and present in amounts such that the silicon structure is ductile.

The silicon structure may exhibit a total permanent strain up to fracture of greater than 5%.

The silicon structure may comprise a unitary body that exhibits a total permanent strain up to fracture of greater than 5%.

The silicon structure may be ductile. The silicon structure may comprise a unitary body that is ductile.

For the absence of doubt, the term “ductile”, used throughout this specification, should be taken as having an identical meaning to that of the term “malleable”, and for the purposes of this specification, a ductile material is defined as a material that exhibits a total permanent strain up to fracture of greater than 3%.

Electrical contact between the metallic component and the silicon component may allow the silicon structure to completely resorb when it is placed in a physiological environment. This may be the case even if the metallic component does not normally, in the absence of electrical contact with the silicon component, resorb when implanted or ingested by human or animal subject.

The silicon structure need not completely resorb, when administered to an animal or human subject, to be of medical value. For example, if it comprises a nanocomposite, then the silicon may resorb leaving nanoparticles of the metal. Provided that the metal is non-toxic, then the nanoparticles may safely be excreted by the body of the subject.

The metallic component may comprise non-toxic nanoparticles, each of the non-toxic nanoparticles comprising one or more of: iron, silver, and gold.

The silicon structure may comprise a metal silicon alloy, the silicon component forming part of the alloy and the metallic component forming part of the alloy.

The silicon component may comprise a silicon substrate, and the metallic component may comprise one or more metallic layers, at least part of the or one of the metallic layers being located on at least part of the surface of the silicon substrate.

The silicon component may comprise a silicon fibre, and the metallic component may comprise one or more metallic layers, at least part of the or one of the metallic layers being located on at least part of the surface of the silicon fibre.

The silicon component may comprise a silicon tube, and the metallic component may comprise one or more metallic layers, at least part of the or one of the metallic layers being located on at least part of the surface of the silicon tube.

The composition of the silicon structure and arrangement of both the silicon component and metallic component may be such that the ratio of the external silicon surface area to the external metallic surface area is between 1 and 1000.

For the purposes of this specification, the external silicon surface of the silicon component is a surface of the silicon component that, were the silicon structure completely immersed in pure water and any reaction with the water prevented, would be in contact with the water when in a state of equilibrium. The external metal surface of the metal component is a surface of the metal component that, were the silicon structure completely immersed in pure water and any reaction with the water prevented, would be in contact with the water when in a state of equilibrium.

The composition of the silicon structure and arrangement of both the silicon component and metallic component may be such that the ratio of the external silicon surface area to the external metallic surface area is between 2 and 1000.

The composition of the silicon structure and arrangement of both the silicon component and metallic component may be such that the ratio of the external silicon surface area to the external metallic surface area is between 10 and 1000.

The composition of the silicon structure and arrangement of both the silicon component and metallic component may be such that the ratio of the external silicon surface area to the external metallic surface area is between 1 and 100.

The composition of the silicon structure and arrangement of both the silicon component and metallic component may be such that the ratio of the external silicon surface area to the external metallic surface area is between 10 and 100.

The composition of the silicon structure and arrangement of both the silicon component and metallic component may be such that the ratio of the external silicon surface area to the external metallic surface area is between 20 and 100.

The composition of the silicon structure and arrangement of both the silicon component and metallic component may be such that the ratio of the external silicon surface area to the external metallic surface area is between 0.001 and 1.

The composition of the silicon structure and arrangement of both the silicon component and metallic component may be such that the ratio of the external silicon surface area to the external metallic surface area is between 0.001 and 0.5.

The composition of the silicon structure and arrangement of both the silicon component and metallic component may be such that the ratio of the external silicon surface area to the external metallic surface area is between 0.001 and 0.1.

The composition of the silicon structure and arrangement of both the silicon component and metallic component may be such that the ratio of the external silicon surface area to the external metallic surface area is between 0.01 and 1.

The composition of the silicon structure and arrangement of both the silicon component and metallic component may be such that the ratio of the external silicon surface area to the external metallic surface area is between 0.01 and 0.5.

The composition of the silicon structure and arrangement of both the silicon component and metallic component may be such that the ratio of the external silicon surface area to the external metallic surface area is between 0.01 and 0.2.

The ratio of the external surface area of the silicon component to that of the metallic component may affect the current density at the external silicon and metallic surfaces, and this in turn may affect the rate of any erosion.

The silicon structure may form at least part of a cylindrical wall. The silicon structure may be shaped and arranged such that it forms a cylindrical wall.

The silicon structure may form part of a stent. The silicon structure may form part of catheter.

The silicon structure may substantially consist of the silicon component and the metallic component. The silicon structure may consist of the silicon component and the metallic component.

The silicon structure may comprise a drug. The silicon structure may form part of a drug releasing stent. The silicon structure may comprise porous silicon and a drug, the drug being located in at least some pores of the porous silicon.

For the purposes of the present specification, a bioactive material is a material that is capable of forming a bond with living tissue when implanted in a living human or animal subject; and a resorbable material is a material that is capable of eroding when placed in a physiological fluid of a living human or animal subject. For the purposes of this specification a physiological electrolyte is an electrolyte that may be found in a living animal or human body.

According to a further aspect the invention provides a method of fabricating a silicon structure comprising a metallic component and a silicon component, the method comprising the steps:
(a) melting a sample of silicon;
(b) allowing and/or causing at least some of the molten silicon to flow into at least some of the pores of a porous metal having a melting point that exceeds that of the sample of silicon; and
(c) allowing and/or causing the silicon and porous metal both to reach a temperature T₂ that is less than the melting point of the sample of silicon, thereby yielding the silicon structure.

The porous metal may comprise one or more of: chromium, molybdenum, tantalum, titanium, and tungsten.

By ensuring that there is sufficient difference between the coefficients of thermal expansion of the metal and the silicon, pressure may be applied to the silicon located in the pores of the porous metal, causing silicon having a beta tin crystal structure to be formed. The porous metal may have a Young's Modulus that is greater than that of bulk crystalline silicon. By ensuring that the metal has a sufficiently high Young's Modulus, the pressure generated by the cooling of the silicon structure, may be concentrated in the silicon component. The porous metal may comprise Iron and/or Nickel.

The method may comprise the step (d) of heating the porous metal to a temperature T₁. The step (d) may occur before or after step (a). Steps (b) and (d) may coincide. Step (b) may cause the temperature of the porous metal to rise.

Steps (a), (b), (c), and (d) may be performed in such a manner that the silicon structure comprises silicon having a beta tin crystal structure.

Step (a), (b), (c), and (d) may each be performed in a reducing atmosphere.

T₁ may be between 1412 and 2355° C. T₁ may be between 1420 and 2000° C. T₁ may be between 1450 and 2000° C. T₁ may be between 1600 and 2100° C. T₁ may be between 1700 and 2000° C. T₁ may be between 1500 and 1800° C.

T₂ may be between 0 C. and 100° C.

The porous metal may be cooled between temperatures T₁ and T₂ over an interval of between 30 seconds and 2 minutes.

The porous metal may be cooled between temperatures T₁ and T₂ over an interval of between 1 minute and 10 minutes.

The porous metal may be cooled between temperatures T₁ and T₂ over an interval of between 1 minute and 60 minutes.

The porous metal may be cooled between temperatures T₁ and T₂ over an interval of between 1 hour and 10 hours.

According to a further aspect the invention provides a method of fabricating a silicon structure comprising a metallic component and a silicon component, the method comprising the steps:
(a) heating a sample of porous silicon, having a tetrahedral bonding structure, to a temperature T₃;
(b) introducing a metal into the pores of the porous silicon; and
(c) allowing and/or causing the porous silicon to cool to a temperature T₄ to yield the silicon structure.

Step (b) may comprise the steps: (bi) melting a metal, having a melting temperature lower than the sample of porous silicon; and (bii) allowing and/or causing the molten metal to flow into at least some of the pores of the porous silicon.

Step (b) may comprise the step: (biii) of melting a compound of the metal; (biv) allowing and/or causing the metal compound to flow into at least some of the pores of the porous silicon; and (bv) of decomposing the metal compound to produce the metal at a temperature T₃.

The metal compound may be one or more of a nitrate, an alkoxide, a beta-diketone, and a mixed alkoxide/beta-diketone.

The metal may comprise one or more of aluminium, barium, bismuth, calcium, gallium, gold, indium, magnesium, silver, tin, and zinc.

By ensuring that there is sufficient difference between the coefficients of thermal expansion of the metal and the silicon, pressure may be applied to the silicon, causing silicon having a beta tin crystal structure to be formed. The porous metal may have a Young's Modulus that is greater than that of bulk crystalline silicon. By ensuring that the metal has a sufficiently high Young's Modulus, the pressure generated by the cooling of the silicon structure, may be concentrated in the silicon component. The porous metal may comprise manganese and/or copper.

Steps (a), (b), and (c) may be performed in such a manner that the silicon structure comprises silicon having a beta tin crystal structure.

Steps (a), (b) and (c) may each be performed in a reducing atmosphere.

T₃ may be between 30° C. and 1414° C. T₃ may be between 155° C. and 1300° C. T₃ may be between 200° C. and 1000° C. T₃ may be between 400° C. and 1200° C. T₃ may be between 500° C. and 1400° C. T₃ may be between 500° C. and 900° C.

The porous silicon may comprise one or more of microporous silicon, mesoporous silicon, and macroporous silicon.

The porous silicon may have a porosity between 4% and 90%. The porous silicon may have a porosity between 30% and 90%. The porous silicon may have a porosity between 30% and 70%.

T₄ may be between 0° C. and 100° C.

The step (a) may precede the step (b). The step (b) may precede the step (a). The steps (a) and (b) may be performed at the same time.

The porous silicon may be cooled between temperatures T₃ and T₄ over an interval of between 30 seconds and 2 minutes.

The porous silicon may be cooled between temperatures T₃ and T₄ over an interval of between 1 minute and 10 minutes.

The porous silicon may be cooled between temperatures T₃ and T₄ over an interval of between 1 minute and 60 minutes.

The porous silicon may be cooled between temperatures T₃ and T₄ over an interval of between 1 hour and 10 hours.

According to a further aspect, the invention provides a silicon structure as defined in any of the above mentioned aspects for use as a medicament.

According to a further aspect, the invention provides a silicon structure as defined in any of the above mentioned aspects for use as a biomaterial.

According to a further aspect, the invention provides a silicon structure as defined in any of the above mentioned aspects for use as a bioactive material.

According to a further aspect, the invention provides a silicon structure as defined in any of the above mentioned aspects for use as a resorbable material.

According to a further aspect, the invention provides a silicon structure as defined in any of the above mentioned aspects for use as a resorbable biomaterial.

According to a further aspect, the invention provides a silicon structure as defined in any of the above mentioned aspects for use as a stent.

According to a further aspect, the invention provides a stent comprising a silicon structure as defined in any of the above mentioned aspects.

According to a further aspect the invention provides a membrane comprising a silicon structure as defined in any of the above mentioned aspects.

According to a further aspects the invention provides a fibre comprising a silicon structure as defined in any of the above mentioned aspects.

The invention will now be described, by way of example only, with reference to the following diagrams:

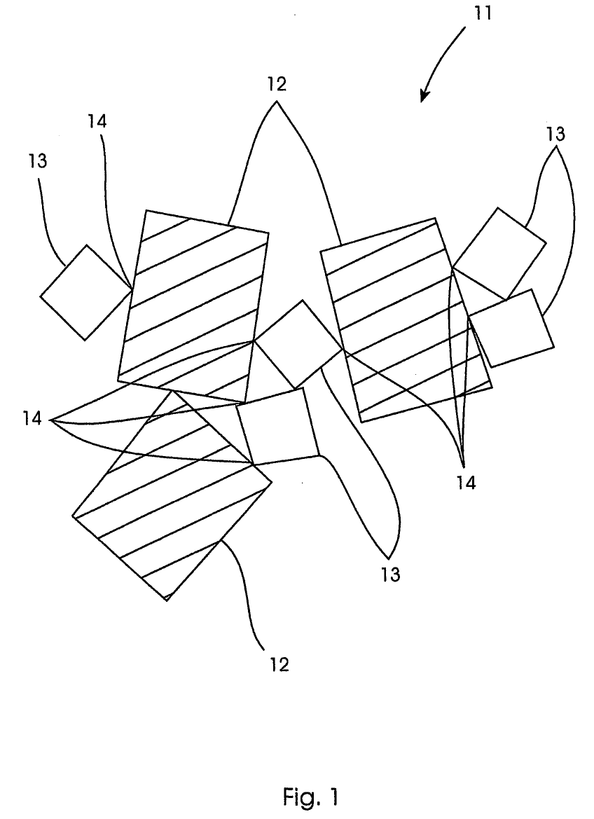

FIG. 1 shows a schematic diagram of part of a silicon structure, according to the present invention, which comprises a nanocomposite of a metallic component and a silicon component;

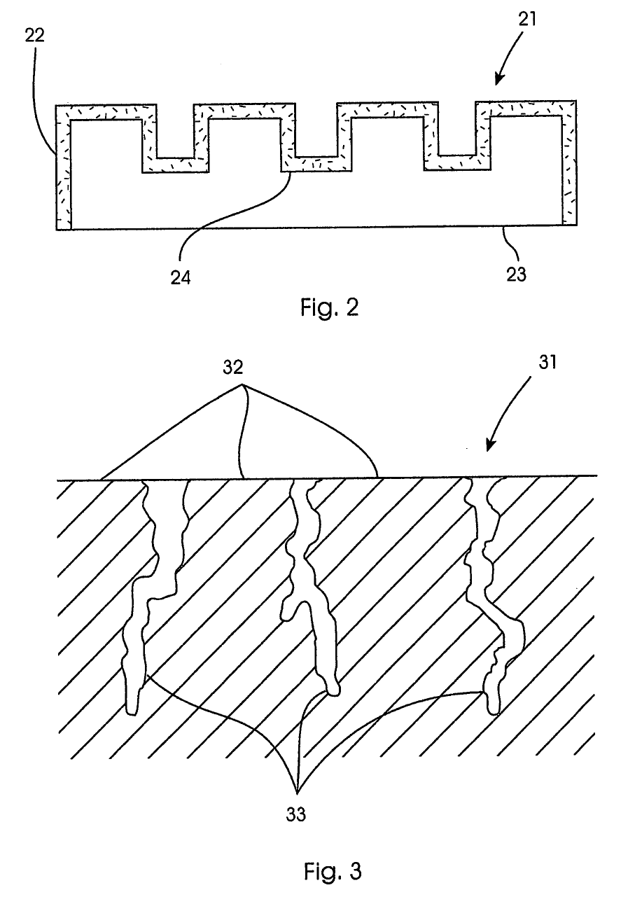

FIG. 2 shows a schematic diagram of a silicon structure having an external metallic surface area that is greater than the external silicon surface area; and

FIG. 3 shows a schematic diagram of part of a silicon structure, comprising porous metal and silicon having a beta tin structure, the silicon being located in the pores of the metal.

FIG. 1 shows a schematic diagram of part of a silicon structure generally indicated by 11, which comprises a nanocomposite of a metallic component and a silicon component. The metallic component comprises a metal that is more noble than silicon. The metallic component comprises a multiplicity of metallic nanoparticles 12, and the silicon component comprises a multiplicity of silicon nanoparticles 13. In FIG. 1, electrical contacts 14 have been formed between the metallic and silicon nanoparticles 12, 13. Because of the difference between the electrochemical properties of the metallic and silicon nanoparticles 12, 13, when the silicon structure 11 is immersed in a physiological liquid a potential difference is established between the silicon nanoparticles 13 and the metallic nanoparticles 12; the potential difference increasing the rate of erosion of the silicon nanoparticles 13.

A silicon structure, according to the invention, comprising a metallic component and a silicon component may be fabricated by one or more of the following standard methods: liquid metal infiltration, squeeze casting, stir casting, hot isostatic pressing (HIP), cold isostatic pressing (CIP), chemical vapour deposition within a metal preform, and liquid organometallic impregnation of a silicon preform followed by pyrolysis.

Liquid metal infiltration may comprise the step of immersing a porous silicon preform in a molten metal contained within a suitable furnace. A hydrostatic pressure of between 10 and 1,000 atmospheres may be applied to the metal to improve wetting of the porous silicon by the metal. The general technique is described in J. Appl. Phys. 64(11) p 6588-6590 (1988) which is herein incorporated by reference.

Hot isostatic pressing may comprise the step of loading silicon particles and metal particles into a hot isostatic press, the press may be located in a furnace enclosed in a water cooled autoclave, the furnace being used to heat the mixture of metal and silicon particles, pressure may be applied to the heated particles by means of argon or helium gas. The general technique is described in U.S. Pat. No. 5,919,321 and in J. Mater. Sci. 31,4985-4990 (1996), both of which is herein incorporated by reference.

The Cold Pressing may comprise the step of loading silicon particles and metallic particles into a stainless steel cylinder, a uniaxial pressure between 1,000 and 5,000 psi may then be applied by means of a stainless steel piston, the temperature of the silicon and metal particles being maintained at approximately 20 C. The use of CP to consolidate a particulate product may be carried out prior to HIP to obtain the desired composite.

Magnetron sputtering may comprise the step of physical vapour deposition of the silicon and metallic components to generate a multilayer nanocomposite structure. The general technique is described in SPIE Vol. 3331, 42-51 (1998) which is herein incorporated by reference.

Silane decomposition may comprise the step of chemical vapour deposition of the metallic component to generate the composite from the porous silicon preform. Deposition temperatures are typically between 550 C. and 650 C., deposition occurring in a low pressure (0.2-1.0 torr) reactor. The technique is described in VLSI Technology by S. M. Sze, McGraw Hill 1998.

Further standard techniques by which a nanocomposite, comprising a metallic component and a silicon component, may be fabricated include: milling (described in J. Alloys and Compounds Vol. 264, 285-292 (1998) which is herein incorporated by reference), co-sputtering (described in Thin Solid Films 320, 184-191 (1998) which is herein incorporated by reference), thermally induced phase separation (described in Materials Science end Engn. Vol. B100, 27-34, 2003, which is herein incorporated by reference), and electrodeposition from the liquid phase into a porous preform (described in J. Appl. Phys. Vol. 76, p 6671-2, 1994, which is herein incorporated by reference).

FIG. 2 shows a schematic diagram of a silicon structure, generally indicated by 21, comprising a metallic component 22 and a silicon component 23. The external metallic surface area is greater than the external silicon surface area. This is because the metallic electrode 22 has a corrugated surface, whereas that of the silicon substrate 23 is flat. The metallic electrode may be formed by first micromachining the silicon substrate, so that corrugations are formed in its non-external surface 24. The metallic layer is then deposited upon the non-external surface 24 to form the corrugated metallic electrode 22.

The ability to alter the ratio of the metallic external surface area to the silicon external surface area is of great value in controlling the rate of etching or corrosion of the silicon when the silicon structure is placed in the an electrolyte such as a physiological electrolyte. The greater the surface area of the metallic electrode 22, the greater the current density resulting from the potential difference between the metallic electrode 22 and the silicon substrate 23, and the greater the rate of etching or erosion.

FIG. 3 shows a schematic diagram of part of a silicon structure, generally indicated by 31, comprising a porous metal component 32 and a silicon component 33 having a beta tin structure, the silicon 33 being located in the pores of the metal.

A silicon structure comprising a metallic component and a silicon component having a beta tin crystal structure may be fabricated by taking a porous metal preform and introducing molten silicon into the pores of the preform. The preform may be produced by partial consolidation of metallic powder. Sintering is carried out to achieve a porosity of between 10 and 50%. The silicon is heated to a temperature of between 1450 and 1550 C. in a furnace under a reducing atmosphere of hydrogen, and the metallic preform is immersed in the molten silicon. Once the molten silicon has passed into the pores of the porous metal, the structure is removed from the furnace and allowed to cool. The pressure applied by the contraction of the porous metal causes the silicon trapped in the pores to form a beta tin crystal structure. A hot press or a hot isostatic press may be utilized to apply additional pressure to the composite. This method is suitable for fabricating silicon structures comprising chromium, iron, molybdenum, nickel, tantalum, titanium, and tungsten.

A silicon structure comprising a metallic component and a silicon component having a beta tin crystal structure may be fabricated by taking a sample of 50 to 90% porosity mesoporous silicon having a tetrahedral bonding structure, and melting a molten metal salt into the pores of the porous silicon by the method described in WO 99/53898 which is herein incorporated by reference. Decomposition to the metal is carried out in a reducing atmosphere of hydrogen between 800 and 1200 C. The cooling of the metal exerts a pressure on the silicon causing its structure to change to the beta tin form. This method is suitable for the fabrication of silicon structures comprising one or more of the following metals: aluminium, barium, bismuth, calcium, copper, gallium, gold, indium, magnesium, manganese, silver, tin, and zinc. The metal salt used in this process may be selected from one or more of a nitrate, an alkoxide, a beta-diketone, and a mixed alkoxide/beta-diketone.

In a further example, a silicon structure according to the present invention may be fabricated by taking commercially supplied micronized bulk silicon powder RG98 powder and treating it with a 1:1 solution of HF and ethanol. The powder may be left in the solution for 5-15 minutes, to remove surface oxide arising from atmospheric exposure. The cessation of gas evolution may be taken as an indication that hydride surface formation is complete. Extraction of the silicon powder may be achieved with Buckner vessel filtration, followed by air drying on filter paper. Equal weights of a metal powder (for example Fe powder, W powder, Mo powder, or Ni powder) and the silicon powder may then be mixed using a Pharmatech Ltd LC005 blender, operating at 28 rpm for 60 minutes. Some of blended powder may then be accurately weighed, prior to loading into the evacuable pellet die, ready for cold pressing. Samples may then be cold pressed in a Rondal 10 tonne hydraulic press at a range of different pressures to produce the silicon structure.

In a further example, a silicon structure according to the present invention may be fabricated by taking micronized powders of both metal and silicon and subjecting them to communition via ball milling. Both the metallic and silicon components may be comprise part of a slurry. Zirconia microspheres, having a diameter between 1 and 200 micons, may be used to thoroughly mix the components. The Netzsch Zeta II LMZ 10 Circulatory Mill may be employed to perform this task. The mixed silicon and metallic components may then be cold pressed in the Rondal hydraulic press, as described above, to yield the silicon structure.

A silicon structure according to the present invention may also be fabricated by taking commercially available nanoparticles of metal and silicon and mixing them with supercritical CO₂ as described in “Mixing of Nanoparticles by rapid expansion of high pressure suspensions” by J Yang, J Wang, R N Dave, R Pfeffer in Advanced Powder Technology Vol 14 no 4, (2003) pp 471-493, which is herein incorporated by reference.

The silicon structure fabricated by one or more of the abovementioned techniques may be implanted into an animal or human subject by standard surgical techniques.

1-7. (canceled)

8. A silicon structure for use as a resorbable biomaterial, the silicon structure comprising a metallic component and a porous silicon component, at least some of the metallic component being arranged in at least some of the pores of the silicon component, and in electrical contact with at least part of the silicon component, the silicon component comprising microparticulate silicon and the metallic component comprising a microparticulate metal that is more noble than silicon.

9. A silicon structure according to claim 8 characterised in that the metallic component comprises one or more of: gold, platinum, silver, palladium, selenium, copper, bismuth, tungsten, molybdenum, nickel, and iron.

10. A silicon structure according to claim 8 characterised in that the silicon component comprises nanostructured silicon and the metallic component comprises nanocrystalline metal.

11. A silicon structure according to claim 8 characterised in that the structure comprises a unitary body, the unitary body comprising at least part of the silicon component and/or at least part of the metallic component.
